(19) United States
(12) Patent Application Publication
(10) Pub. No.: US 2013/0023785 A1
(43) Pub. Date: Jan. 24, 2013

(54) Probe for oral thermometer

(71) Applicant: Jacob Fraden, San Diego, CA (US)

(72) Inventor: Jacob Fraden, San Diego, CA (US)

(21) Appl. No.: 13/135,981

(22) Filed: Jul. 21, 2011

(51) Int. Cl. A61B 5/01

(52) U.S. Cl. 600/549

(57) ABSTRACT

A shape of the oral temperature probe is sculptured to facilitate its self-guidance toward the root of the tongue. The probe body consists of two distinct sections—the stem and curved elbow. The stem carries a contact temperature sensing tip. The elbow allows the probe to go around the teeth of the lower jaw and position the stem under the tongue in order to direct the temperature sensing tip toward the root of the tongue. To speed up the probe temperature response, before inserting the probe into a mouth, the probe tip is preheated to a temperature that is cooler than the lowest expected temperature of the patient.

### FIELD OF INVENTION

This invention relates to devices for measuring temperature, more specifically to medical thermometers.

### DESCRIPTION OF PRIOR ART

A contact medical thermometer is a device capable for measuring temperature by means of a physical contact with the object of measurement. Medical thermometers are known in art for over 150 years. Their designs range from a glass tube filled with liquid as exemplified by U.S. Pat. No. 3,780,586 issued to Donofrio, to liquid crystal probes as exemplified by U.S. Pat. No. 4,779,995 issued to Santacaterina et al, to a plastic tube with a metal sensing tip as exemplified by U.S. Pat. No. 4,813,790 issued to Frankel et al. All the above patents are incorporated herein as references.

Depending on medical reasons and cultural preferences, noninvasive temperature from a patient is typically measured by an axillary (under-the-armpit) probe, by an oral probe in a sublingual pocket, by a rectal probe in the anus, by an ear canal infrared probe, by a contact probe behind the ear lobe, by an infrared probe from the forehead, etc. Invasive (internal) temperatures are measured by an esophageal probe, a Swan-Ganz catheter, etc. In all cases, for a quality measurement it is essential to achieve a good thermal coupling between the temperature sensor inside the probe and the patient's body site of measurement, for example, the forehead skin or sublingual tissue. This invention concerns with an oral probe. The probe is part of a contact thermometer that shall come in a physical contact with the sublingual tissue in the mouth of a patient, preferably in the vicinity of a sublingual artery.

Due to a possible talking, sneezing, coughing and breathing by a patient, many areas in the mouth, even in a sublingual pocket, may have lower temperatures than that of the inner (core) body. It can be shown anatomically that the best place for the oral temperature measurements is the area in a mouth under the tongue where the sublingual artery passes near the root of a tongue. This area has a stable temperature because it's well thermally shielded from the outside and is closer a carotid artery.

FIG. 1 illustrates a typical oral probe 1 of the prior art. It has a shape of an elongated stem 2. A temperature sensor is positioned inside the tip 6. For the sanitary purposes, stem 2 may be covered by a protective probe cover 5 having low thermal resistance, at least in vicinity of the tip 6. At the opposite side, stem 2 is supported by a handle 3. A temperature related signal from the tip 6 is communicated to the thermometer processing circuit (not shown in FIG. 1) via a conductor 4. When the probe 1 is inserted into mouth of a patient, it may be placed on the top or under the tongue. Thus, the tip 6 may or may not come into a good thermal coupling with a sublingual tissue. This greatly depends on the patient cooperation for placing the probe sufficiently deep under the tongue.

To improve a thermal coupling between the temperature sensitive tip and the sublingual pocket tissues, a resilient pacifier probes were proposed as exemplified by U.S. Pat. No. 5,176,704 issued to Bernd and a flexible probe as taught by U.S. Pat. No. 5,013,161 issued to Zaragoza et al. The probe has a bend to facilitate a better thermal contact with the sublingual area as taught by the U.S. Pat. No. 7,036,984 issued to Penney et al. Another embodiment with a bent probe is taught by the U.S. Pat. No. D525,542 issued to Russak et al. All the above patents are incorporated herein as references.

FIG. 2 illustrates a prior art thermometer produced by Timex. It incorporates a curved probe to facilitate an intuitive placement of the sensing tip 6 under the tongue. This is a noticeable improvement over the prior art probe shown in FIG. 1. The probe of FIG. 2 has a bend 9 being attached to the thermometer housing extension 3. The housing 20 incorporates a power supply, processing circuit, display 21 and may contain some switches, for example power switch 27. Although this prior art probe, when placed in a mouth, forces the tip 6 for an intuitive positioning under the tongue, it fails to resolve a problem of an intimate thermal coupling between the tip 6 and a sublingual artery area.

A speed response is a major issue with any contact thermometer and with the oral thermometers specifically. When a colder probe (initially at a room temperature, e.g.) is placed into the patient mouth, it alters the oral tissue temperature so much that a substantial time is required to re-warm the oral tissue to the pre-insertion temperature level. Typically, this time may range from 6 seconds to a minute. If the re-warming time is ignored, accuracy is compromised. One way to minimize a thermal drag by a cooler probe is to pre-warm the probe to a temperature that is substantially close to the oral anticipated temperature. This approach is exemplified by a U.S. Pat. No. 5,632,555 issued to Gregory et al. and U.S. Pat. No. 6,109,784 issued to Weiss. The above patents are incorporated herein as references.

The prior art oral probes have several drawbacks, such as a poor coupling between the probe and the root of a tongue. A poor coupling reduces accuracy and prolongs the measurement time. Another limitation of the prior art that teaches the heated oral probes is a need for a manual initiation of the measurement upon inserting the probe into the patient's mouth. And another limitation is placing the probe in a wrong spot inside the mouth by an inexperienced operator.

Thus, the goal of this invention is to offer an oral temperature probe that would facilitate an intuitive self-guidance of the probe tip toward the root of the tongue, when placed in the mouth.

An additional goal of the invention is to increase a thermal contact between the probe temperature sensor and the root of a tongue.

Another goal of this invention is providing a fast speed response of the probe,

And additional goal is to make the oral thermometer operation requiring a minimal control by the operator. Attainment of these and other goals will be apparent from the foregoing description of the invention.

### SUMMARY OF INVENTION

A shape of the oral temperature probe is sculptured to facilitate its self-guidance toward the root of the tongue. The probe body consists of two distinct sections—the stem and curved sections. The curved section allows the probe to go around the teeth of a lower jaw and to position the stem section under the tongue in order to direct the temperature sensing tip toward the root of the tongue. To speed up the probe temperature response, before inserting the probe into a mouth, the probe tip is preheated to a temperature that is cooler than the lowest expected temperature of the patient.

### BRIEF DESCRIPTION OF DRAWINGS

FIG. 1 illustrates a prior art oral probe having a straight shape.

FIG. 2 illustrates a prior art oral probe having a curved shape.

FIG. 3 shows the oral probe having curved and straight sections.

FIG. 4 illustrates a two-section oral probe being attached to a thermometer body.

FIG. 5 is a view of a two-section oral probe inserted into a sublingual pocket of a patient.

FIG. 6 shows three cross-sectional views of the stem.

FIG. 7 illustrates a cross-sectional view of the probe tip with a temperature sensor and heater.

FIG. 8 shows an isometric view of the sensing shell.

FIG. 9 is a timing diagram of the thermometer operation.

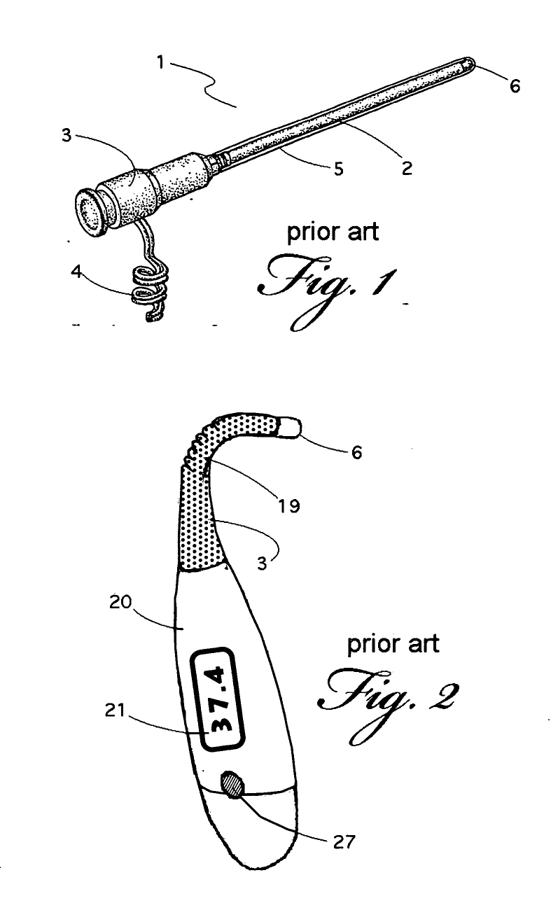

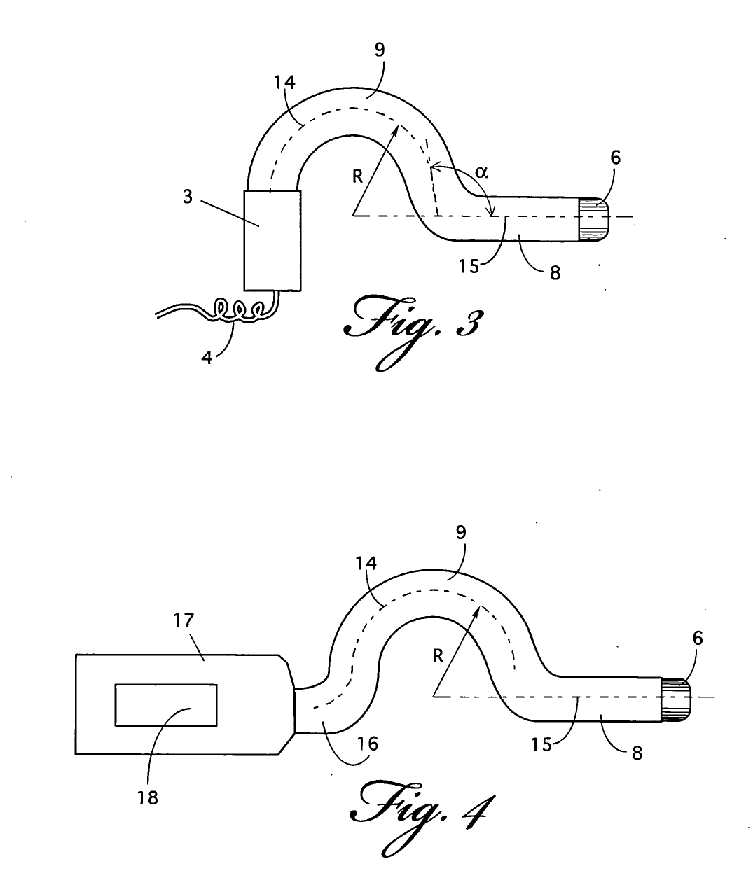

### DESCRIPTION OF PREFERRED EMBODIMENTS

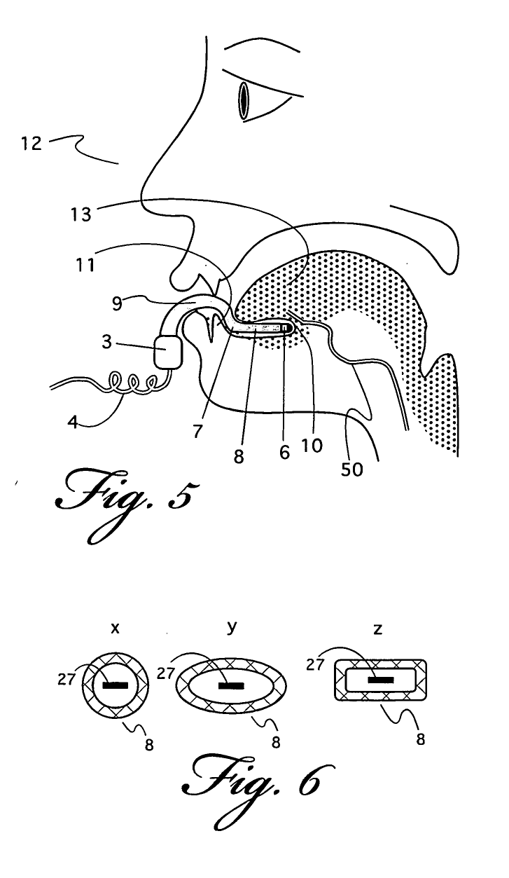

The new and improved probe for an oral contact thermometer is illustrated in FIG. 3. It is comprised of two distinct sections: the curved section which is the elbow 9 and the straight section which is the stem 8. Both sections are joined together in a smooth and continuous manner that the curved axis 14 of elbow 9 and the straight axis 15 of stem 8 are mutually disposed at a joint angle a ranging from 90° to 135°. The stem 8 at its distal end carries the tip 6 that among other components incorporates a temperature sensor. In one embodiment the proximal end of elbow 9 is terminated at a handle 3 which may be connected to the conductor 4 whose purpose is to transmit signals from the tip 6 to an external data acquisition equipment, such a processing circuit. Alternatively, elbow 9 via the neck 16 may be attached to the body of a thermometer 17 as shown in FIG. 4. Such a thermometer may comprise numerous additional components, like a power supply, signal processing circuit, control switches and an output device, for example a display 18 that shows the value of patient temperature.

A typical radius R of the curved axis 14 ranges between 10 and 25 mm. This was selected to accommodate sizes of the human teeth and lower jaws. In some designs, it may be desirable to make radius R variable, that is to fabricate elbow 9 of a pliant material capable of retaining its shape after been manually bent to increase or decrease radius R. When radius R changes, the joint angle a will also change.

Length of stem 8 is selected to assure that when placed in the mouth under the tongue 13 (see FIG. 5), tip 6 will touch the sublingual surface 10 that is in the close proximity to the sublingual artery 50 of patient 12. The elbow 9 wraps around teeth 11 and forces stem 8 to slide under the tongue 13. For most practical purposes, the stem length should be between 10 and 30 mm. Thanks to the combination of a curved and straight sections, the tip 6 is automatically placed correctly under the tongue to come into an intimate thermal coupling with the sublingual artery tissue. Any other (wrong) probe placement, such as over the tongue, is uncomfortable and would require an extra effort by the patient which usually is not the case in medical practice.

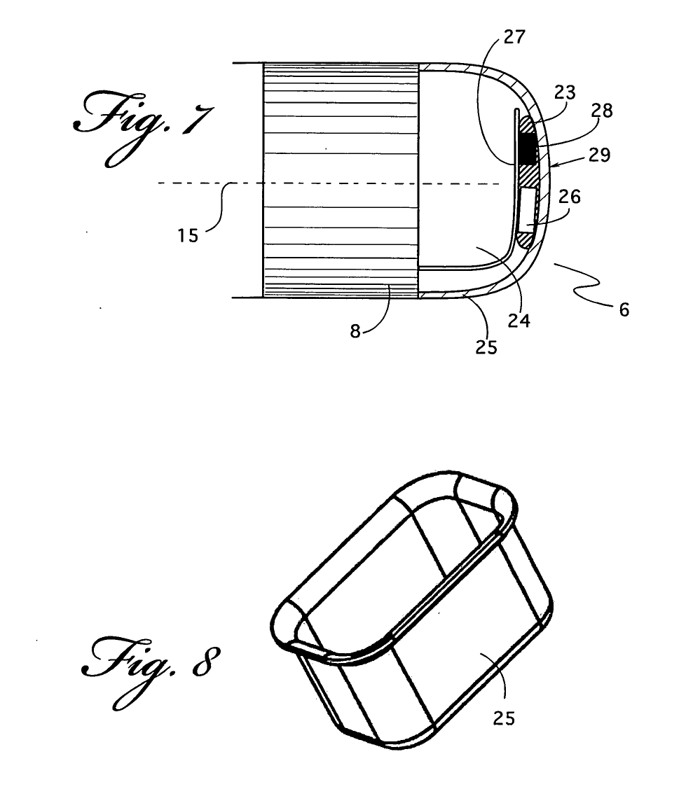

Both sections of the probe, elbow 9 and stem 8, should be fabricated of a material having low thermal conductivity and easy cleanable. An example of the material is ABS resin. A cross-sectional profile of the probe may have any practical shape—round, oval, rectangular, etc. This is illustrated in FIG. 6 for the round (x), oval (y) and rectangular (z) cross-sections of the stem 8. The same consideration is applicable for cross-sections of the elbow 9.

As it was indicated above, tip 6 comprises a temperature sensor. Examples of such sensor are a thermistor, thermocouple junction, resistive temperature device (RTD) and semiconductor p-n junction.

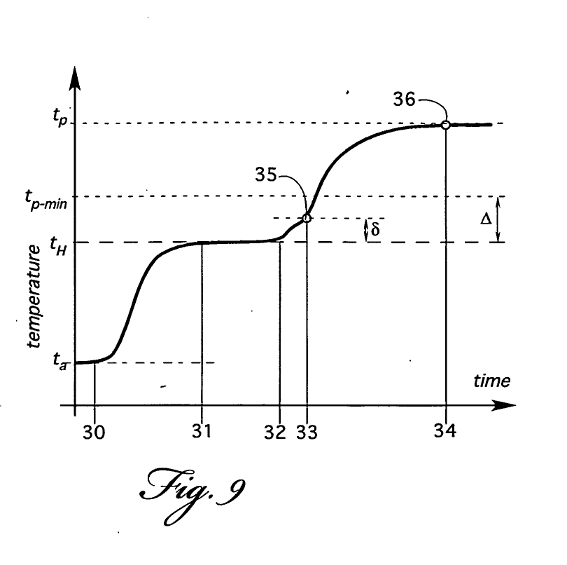

The outer shell 25 of the tip 6 (FIG. 7) is fabricated of a material having high thermal conductivity, preferable metal, such as aluminum, copper or brass. Thickness of the shell 25 is between 0.1 and 0.5 mm. The isometric view of the metal shell is illustrated in FIG. 8. The outers side 29 of the shell 25 is intended for contacting the patient sublingual tissue and thus preferably should be given a protective coating, for example anodizing or gold plating. Any plating or coating must be thin (<5 micrometer). If needed, a probe cover of a conventional design known in art may be placed over the stem 8 or the entire probe.

FIG. 6 illustrates tip 6 that in addition to the temperature sensor 28, comprises a heating element 26. The heating element may be needed to shorten the tip 6 time of response as explained below. The temperature sensor 28, heating element 26 (if present) and the shell 25 must be in an intimate thermal coupling with each other. This is accomplished by a holding media 23 that may be a thermally conductive epoxy or solder. The media 23 holds these components together and provides a thermal coupling. Electric connections with an external circuit (not shown) to the components inside the tip 6 are provided by a set of conductors 27, for example a flex circuit board fabricated on a polyimide substrate. The inner space 24 of the tip 6 preferably should be void of any other material (with a possible exception of air), thus a thermal coupling between the temperature sensor 28 and other components positioned outside the tip 6 will be minimized and an overall thermal mass of the tip will be low enough for a fast response to temperature changes.

The heating element 26 should be turned on/off in a prescribed manner. Also, a signal produced by the temperature sensor 28 should be processed in a specific timing relationship with the heater 26 operation. FIG. 9 is temperature-time graph that illustrates the preferred relationships between various temperatures of the tip 6 during thermometer operation for the case when the probe temperature tₐ before the measurement is substantially lower than the minimal anticipated patient temperature tₚ₋ₘᵢₙ. At first, the tip 6 has initial temperature tₐ that may be a room temperature, for example 20° C. The actual patient oral temperature in a sublingual pocket at a root of the tongue is tₚ, for example 39.4° C. The lowest anticipated temperature of the patient oral body site is tₚ₋ₘᵢₙ, for example 34° C.

At the first instant 30, the thermometer is turned on and the electronic control circuit starts supplying electric power to the heater 26 to elevate its temperature to the predetermined level of a pre-warmed temperature t_{H}. This set temperature t_{H} of the heater is close and preferably lower than the lowest anticipated patient temperature tₚ₋ₘᵢₙ=34° C. For example, we may select t_{H}=33° C. For most practical purposes, the offset Δ between these two temperatures should be 0.5-2.0° C. When the temperature sensor 28 reaches the pre-warmed temperature set t_{H} at the second instant 31, this temperature of the tip 6 is stabilized and maintained by the feedback control loop of the electronic circuit for as long as needed to place the probe into the mouth of the patient.

The operator places the probe into the patient's mouth so that a tip 6 of the probe is pressed against the root of the tongue at the third instant 32. This quickly elevates the sensor 28 temperature above the t_{H} level. This “jump” in temperature is detected by the electronic circuit when its value 35 reaches the predetermined threshold at the fourth instant 33. Note that the jump threshold value (t_{H}+δ) should be less or equal to the lowest possible oral temperature tₚ₋ₘᵢₙ.

At this fourth instant 33, electric power to the heater 26 is turned off and temperature of the tip 6 is allowed to evolve to the actual patient temperature tₚ, which is reached at the fifth instant 34 when the tip temperature has the end value 36. At this fifth instant 34 the tip 6 and the sublingual surface 10 are in a thermal equilibrium, the measurement is over and the end value 36 temperature or its equivalent signal is sent to the output element 18, for example a display. Since the temperature t_{H} is much closer to the final temperature tₚ than the initial temperature tₐ, the measurement time (between instances 32 and 34) is drastically reduced. Experimentally it was shown the time is between 1 and 3 s.

One of the important innovations of this invention is control of electric power supplied to the heart by a “jump” is a signal communicated by the temperature sensor. This innovation allows for an automatic detection of the probe placement in the mouth and thus eliminates a need for a manual control of the temperature taking cycle.

In cases when the initial temperature tₐ is already warm, meaning it's equal or higher than the heater set temperature t_{H}, the heater is never turned on and the cup 6 allowed to equilibrate with the patient tₚ temperature, just as in the conventional equilibrium thermometers known in art.

In other embodiments, the heater 26 is not employed and no probe pre-warming performed. Then, the measurement time is either accepted as being slower or it may be shortened by some other methods, such as one of several predictive algorithms known in art. A predictive algorithm predicts or anticipates patient temperature from a rate of change in temperature of a temperature sensor. Yet, even without a heating or prediction, a probe that is the subject of this invention would still provide a more accurate measurement due to more consistent and reliable coupling between the tip and the patient at the root of the tongue.

The invention has been described in connection with preferred embodiments, but the invention is greater than and not intended to be limited to the particular form set forth. The invention is intended to cover such alternatives, modifications, and equivalents as may be included within the spirit and scope of the invention as defined by the appended claims.

1. A probe of a thermometer for insertion into a mouth of a patient for measuring patient temperature, comprising
a stem disposed along a straight axis, the stem having a proximal end and a distal end;
a tip attached to the distal end of the stem and comprising a sensor responsive to temperature and thermally coupled to the tip;
a joint portion attached to the proximal end of the stem at a first joint end of the joint portion and forming a convex arc from the straight axis; and
an elbow attached to a second joint end of the joint portion and forming a concave arc to the straight axis, the elbow and joint portion positioning the tip at a sublingual tissue by a sublingual artery.

2. The probe of a thermometer of claim 1, wherein the convex arc forms a joint angle with a value between 90 and 135 degrees.

3. The probe of a thermometer of claim 1, wherein the tip comprises a heating element thermally coupled to the sensor.

4. The probe of a thermometer of claim 1, wherein the stem has a length between 10 and 30 mm measured from the proximal end to the distal end.

5. The probe of a thermometer of claim 1, wherein the elbow has a radius of curvature ranging from 10 mm to 25 mm.

6. The probe of a thermometer of claim 1, where the tip comprises an outer shell fabricated of metal and the stem comprises a flat wall normal to the straight axis, the flat wall having an outer side contacting the patient and an inner side in communication with the sensor.

7. The probe of a thermometer of claim 1, wherein the stem and the elbow are fabricated of a material having lower than metal thermal conductivity.

8. The probe of a thermometer of claim 3, wherein electric power is provided to the heating element from the thermometer for bringing a temperature of the heating element to a set temperature having value equal or below an anticipated lowest oral temperature of the patient by a predetermined offset.

9. The probe of a thermometer of claim 8, wherein the electric power is configured to be disconnected after a temperature that is measured by the sensor exceeds the set temperature by a predetermined threshold.

10. The probe of a thermometer of claim 1, wherein the measurement of patient temperature is anticipated from a rate of change in temperature measured by the sensor.

11. (canceled)

12. (canceled)

13. (canceled)

14. A medical thermometer for measuring a temperature of a patient, comprising:
a housing;
an electronic circuit;
an output device;
a probe comprising:
a stem disposed along a straight axis, the stem having a proximal end and a distal end;
a tip attached to the distal end of the stem and comprising a sensor responsive to temperature and thermally coupled to the tip;
a joint portion attached to the proximal end of the stem at a first joint end of the joint portion and forming a convex arc from the straight axis; and
an elbow attached to a second joint end of the joint portion and forming a concave arc to the straight axis, the elbow and joint portion positioning the tip at a sublingual tissue by a sublingual artery.

15. The medical thermometer of claim 14 wherein the elbow is connected to the housing, the tip comprises a metal shell thermal coupled to the sensor, and
the sensor generates a signal representative of the temperature.

16. The medical thermometer of claim 14 wherein the tip further comprises a heating element thermally coupled to the sensor and receiving electric power from the electronic circuit.

17. The medical thermometer of claim 15 wherein the signal is sent to the electronic circuit for computation of the temperature of the patient, wherein the electronic circuit sends a computed temperature to said output device.

18. The medical thermometer of claim 17 wherein the electronic circuit computes the temperature by using a rate of change in the signal.

19. The probe of a thermometer of claim 6 wherein the outer shell has an outer side comprising a flat portion that is disposed normal to the straight axis.

20. The medical thermometer of claim 15 wherein the metal shell and the stem are configured for minimizing a mutual thermal coupling.

21. The medical thermometer of claim 14 further comprising a probe cover for enveloping the stem and at least a portion of the elbow, wherein said probe cover is fabricated of a polymer film.

22. The medical thermometer of claim 15 wherein the metal shell has an outer side adapted to be contacted by the sublingual tissue of the patient and comprising a flat portion normal to the straight axis, the sensor thermally coupled to the outer side.

23. (canceled)

24. A medical temperature probe comprising:
a distal end disposed along a straight axis comprising a sensor responsive to temperature and disposed at a first end of the distal end, the distal end comprising a flat wall normal to the straight axis, the flat wall having an outer side contacting the patient and an inner side in communication with the sensor,
wherein the flat wall is adapted for being positioned at a sublingual tissue by the sublingual artery.

25. The medical thermometer of claim 24, further comprising a heating element in communication with the inner side of the flat wall.
